(19) United States
(12) Patent Application Publication
(10) Pub. No.: US 2006/0173484 A1
(43) Pub. Date: Aug. 3, 2006

(54) Percutaneous breast and buttock modification

(76) Inventor: Stephen Solomon, New York, NY (US)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/317,957

(22) Filed: Dec. 22, 2005

Related U.S. Application Data
(60) Provisional application No. 60/638,565, filed on Dec. 22, 2004.

(51) Int. Cl. A61M 29/00

(52) U.S. Cl. 606/192

(57) ABSTRACT

An apparatus for breast and buttock modification is provided which includes first and second tubes that extend alongside each other, and each of which has a lumen. The apparatus also includes an inflatable balloon having a valve that is detachably coupled to a distal end of the second tube such that a portion of the distal end extends through the valve. The balloon is adapted to be introduced to a space in a body and inflated in the space in the body by fluid introduced through the valve through the second tube. And the second tube is detached from the inflated balloon to leave the inflated balloon positioned in the space in the body.

### CROSS REFERENCE TO RELATED APPLICATION

The present application is based upon and claims the benefit of priority of provisional application Ser. No. 60/638,565, filed on Jun. 23, 2004.

### FIELD OF THE INVENTION

The present invention relates to methods and apparatuses for performing breast and buttock modifications, such as lifts and augmentations, percutaneously.

### BACKGROUND OF THE INVENTION

Breast augmentation, buttock augmentation, breast lifts and buttock lifts are typically performed surgically. More recently, breast augmentation has, for example, has performed with endoscopic guidance.

It should be possible to reduce the risks and costs associated with breast and buttock modification by performing the procedures percutaneously, thereby.

### SUMMARY OF THE INVENTION

According to one aspect of the invention, an apparatus is provided that includes first and second tubes that extend alongside each other, and each of which has a lumen. The apparatus also includes an inflatable balloon having a valve that is detachably coupled to a distal end of the second tube such that a portion of the distal end of the second tube extends through the valve. The balloon is adapted to be introduced to a space in a body and inflated in the space in the body by fluid introduced through the valve through the second tube. And the second tube is detachable from the inflated balloon to leave the inflated balloon positioned in the space in the body.

According to another aspect of the invention, an apparatus for lifting a breast is provided that includes a bone anchor adapted to be screwed into a rib, a tissue anchor including a plurality of barbs to be lodged in breast tissue, and a suture to connect the tissue anchor to the bone anchor. A length of the suture is set to a desired length to lift the breast by a desired amount.

### BRIEF DESCRIPTION OF THE DRAWINGS

FIGS. 1-6 show the placement and inflation of an inflatable balloon for performing breast augmentation;

FIG. 7 is a diagram showing a catheter for introducing and inflating the inflatable balloon;

FIG. 8 is a cross-sectional view along line I-I in FIG. 7;

FIGS. 9-11 show the placement of tissue and bone anchors to perform a breast lift; and

FIG. 12 shows multiple tissue and bone anchor pairs implanted in a breast.

### DETAILED DESCRIPTION

To perform breast augmentation according to the present invention, an inflatable balloon is inserted percutaneously under the pectoral muscle using image guidance, and the balloon is inflated to augment the breast.

More specifically, one technique of performing breast augmentation according to the present invention is shown in FIGS. 1-6.

First, as shown in FIG. 1, a needle 2 such as a Seldinger needle, which includes a sharp stylet inside of a cannula, will be inserted into the breast 100 such that the tip of the needle 1 is posterior to the pectoral muscle 102, but not posterior to the ribs 110. The stylet will then be removed from the cannula 6 of the needle 1.

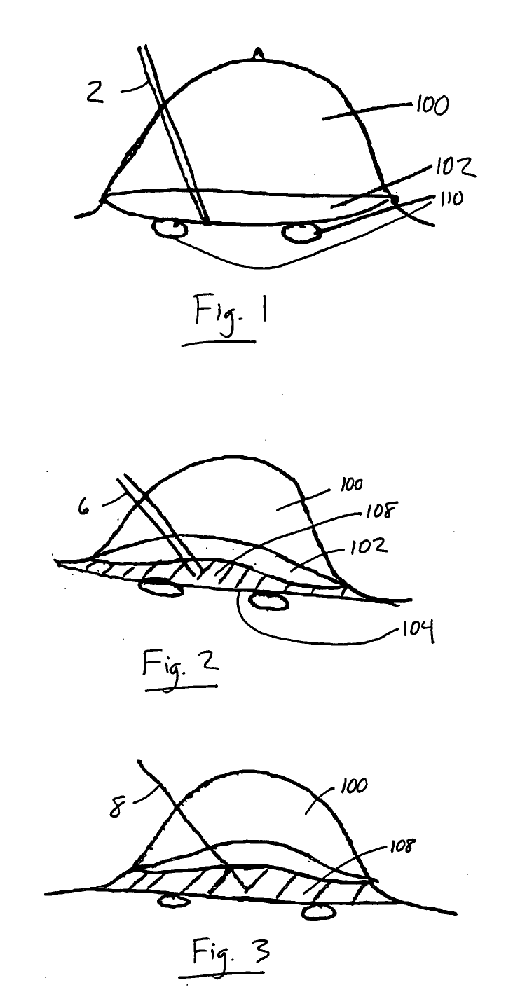

At this stage, fluid (indicated by hatching in FIG. 2), which could include an anesthetic (e.g. lidocaine), may be introduced through the cannula 6 to enlarge a space 108 between the pectoral muscle 102 and the chest wall 104, as shown in FIG. 2. Then, a guide wire 8 will be inserted through the cannula 6 to maintain an access track to the site 108 posterior to the pectoral muscle 102, and the cannula 6 will be withdrawn, as shown in FIG. 3.

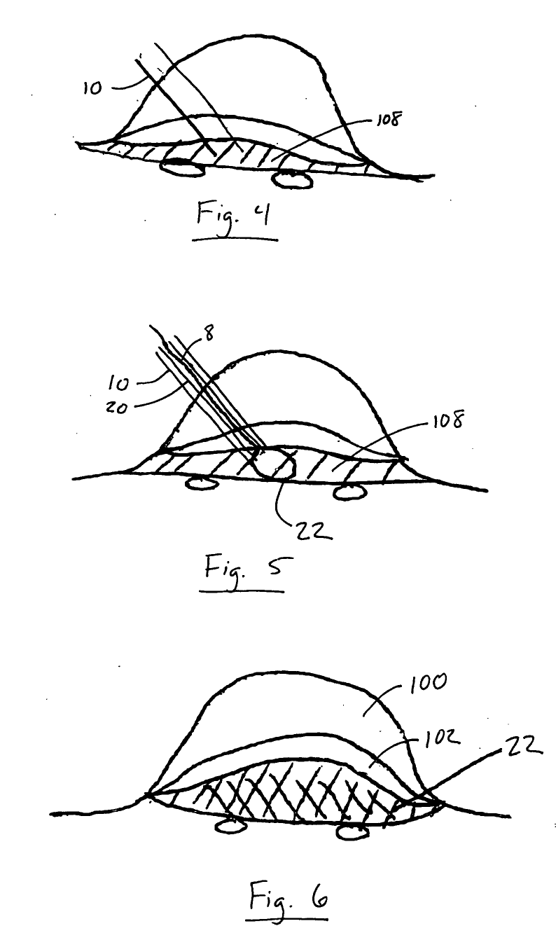

If necessary, the track around the guide wire 8 can be increased in size using a dilator or series of dilators that are slid over the guide wire 8 to increase the size of the track in the tissue using a well-known technique. Once the track to the site 108 has a sufficient size, sheath 10 will be inserted as shown in FIG. 4 through the dilated track to the site 108 posterior to the pectoral muscle.

As shown in FIG. 5, a catheter 20 with a detachable balloon 22 will then be inserted over the guide wire 8 through the sheath 10 to the site 108, and the balloon 22 will then be inflated to a desired size, using a desired fluid. Conventional fluids for inflating breast implants should be suitable for inflating the balloon 22. Once the balloon 22 is sufficiently large, the balloon 22 will be detached and the catheter 20 will be withdrawn. The incision in the breast will then be closed, leaving an inflated implant, as shown by cross-hatching in FIG. 6.

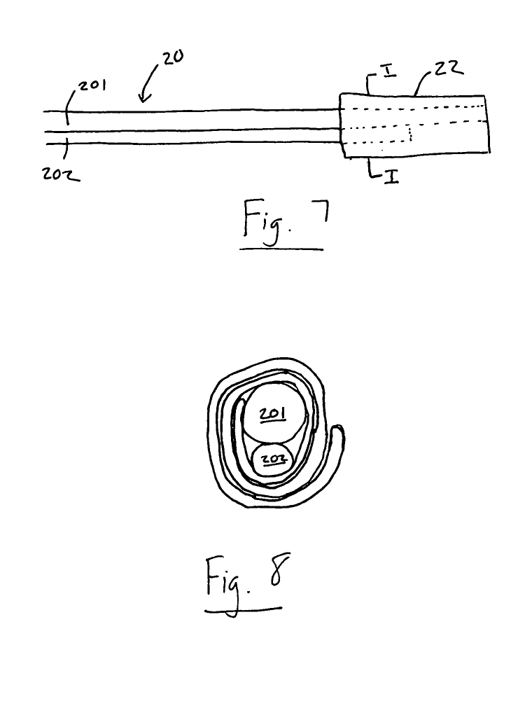

As shown in more detail in FIGS. 7 and 8, the catheter 20 may include a guide catheter 201 that is fed over the guide wire 8 to guide the tip of the catheter 20 to the site 108, and a feeding catheter 202 through which the fluid is fed to the balloon 22. The balloon 22 will be wrapped around the catheter 20 when it is introduced through the sheath 10, and will be unfurled by being inflated. It may be necessary to rotate the catheter 20 to assist in unfurling the balloon 22 as the balloon 22 is inflated.

The balloon 22 is not physically connected to the guide catheter 201, and is coupled to the feeding catheter 202 by a self-sealing valve that closes when the feeding catheter is withdrawn from the balloon 22. Self-sealing valves, for example valves that are formed from elastomeric materials, such as the valve coupling the balloon 22 and feeding catheter 202, are well known in the art, for example for use in inflating balloons for blocking blood vessels with catheters that are detachable from the balloons. For example, self-sealing valves that are detachable balloons are described in U.S. Pat. No. 4,819,637 and U.S. Pat. No. 6,736,793, the entire contents of which are incorporated herein by reference.

It may be beneficial for the feeding catheter 202 to be threaded into the valve portion of the balloon 22, so that the feeding catheter is coupled securely to the balloon 22 while the tip of the feeding catheter 202 extends through the valve to be in position for inflating the balloon 22.

The breast augmentation technique described above may also be applied to buttock augmentation, in which case the balloon should be inserted sub-gluteally.

With the percutaneous breast/buttock augmentation technique described above, the space 108 between the pectoral muscle 102 and the chest wall 104 might be enlarged using an inflatable balloon or series of inflatable balloons, instead of by filling the space 108 with fluid. In addition, catheter 20 and balloon 22 could possibly be inserted over the wire without the use of a sheath 10. Still further, the balloon 22 could be connected to a sub-cutaneous inflation port that could be used to adjust the amount by which the balloon 22 is inflated. If the sub-cutaneous inflation port is used, the necessity of a feeding catheter 202 could be avoided, and the balloon could be deposited in the space 108 and inflated to a desired degree using the inflation port. Yet still further, it might be possible to avoid using the balloon by injecting a hydrogel into the space 108.

According to another aspect of the present invention, a breast lift may be performed by grasping tissue in the breast with a barbed needle and anchoring the barbed needle to a rib with a suture.

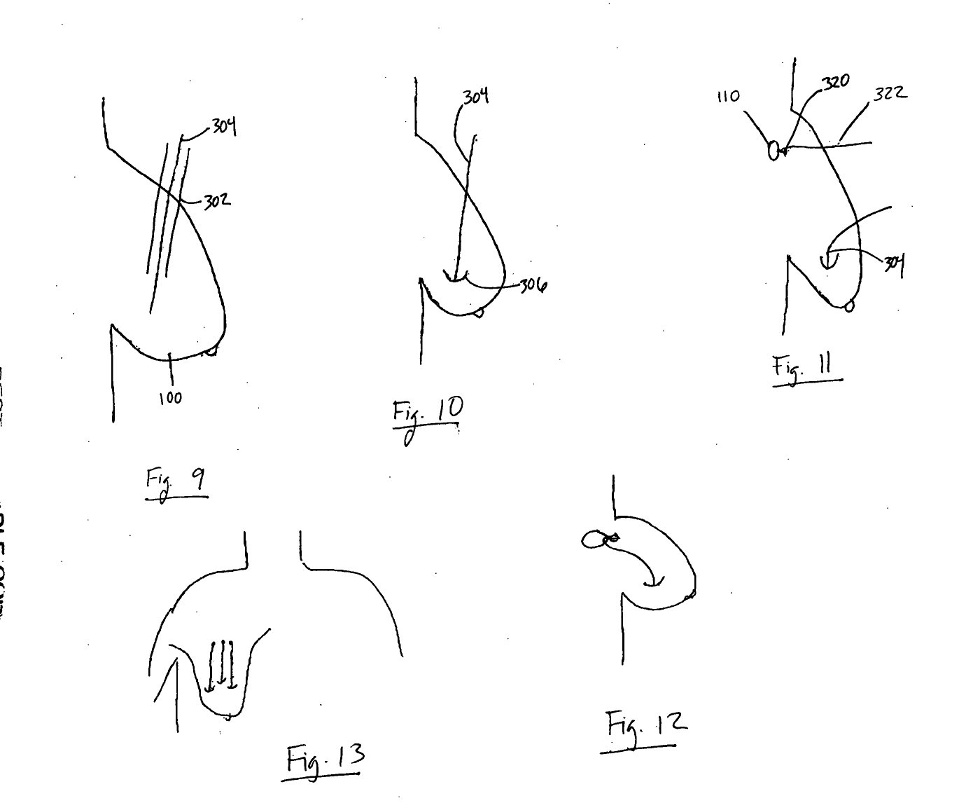

As shown in FIG. 9, a trocar needle 302 will be inserted into the deep subcutaneous fat of breast 100. A tissue anchor needle 304 will be inserted through the trocar 302, and barbs 306 will then deployed from the end of the tissue anchor needle 304, and the trocar 302 will then be removed (FIG. 10). The tissue anchor needle 304 will have enough barbs 306 to securely grab the tissue, and the barbs 306 may expand from the needle 304 in all directions in an umbrella-like array. The tissue anchor needle 304 may have a structure similar to the Semi-Flex RITA Medical RF probes, although with an entirely flexible shaft. The tips of the barbs 306, which may for example be made of nitinol, should not be so sharp that they cut through tissue to the extend that the needle 304 cannot be held in place by the barbs 306.

After (or before; order should make no difference in this technique) the tissue anchor needle 304 is positioned, a bone anchor 320 will be attached to a desired rib 110. The bone anchor 320 has a shape similar to an eye screw, so that a suture can be attached to the bone anchor 320. The bone anchor 320 is introduced to the rib 110 through a sheath that is positioned at the rib in the same way that the sheath 10 described above is positioned so that its distal tip is posterior to the pectoral muscle. That is, a track to the rib 110 is created with a needle and cannula or trocar needle, and the track is dilated and a sheath is inserted to allow access to the rib 110. The bone anchor 320 will then be screwed into the rib 110 with conventional tools, and a suture 322 will be attached to the bone anchor and extend out of the breast 100, as shown in FIG. 11.

The suture 322 will be buried and connected to the flexible shaft (which could also be suture) of the tissue anchor needle 304 by tunneling from the position of the shaft of the tissue anchor needle 304 to the suture 322, or vice versa. Tunneling is a well-known technique for creating a sub-cutaneous path. A tunneling device for creating a path for, for example, a catheter, is described in, for example US 2005/0215994.

The tissue anchor needle 304 will then be attached to the suture 322 attached to the bone anchor, and distance from the barbed end of the tissue anchor needle 304 to the bone anchor will be adjusted to be a desired amount to lift the breast. See FIG. 12. The incisions in the breast will then be closed.

It may be desirable to place multiple tissue anchor/bone anchor pairs in the breast, as shown in FIG. 13. In addition, it may be beneficial for the tissue anchor needle 304 to be coated with, for example, Dacron mesh, so that the tissue grows onto the tissue anchor. Still further, the barbs 306 could be fixed in the deployed position, without the need for a plunger to deploy them. In this case, it may be necessary to dilate the opening through which the tissue anchor needle is inserted, to account for the width of the anchor with the needles deployed.

The procedure described above for performing a breast lift could also be used to perform a buttock lift, by securing the bone anchor to the pelvis.

Presently preferred embodiments of the invention have been described in detail hereinabove. However, various modifications and additions can be made without departing from the spirit and scope of the invention. In particular, the breast and buttock augmentations described above could conceivably be used to augment or lift other parts of the body without departing from the spirit and scope of the present invention. Accordingly, the foregoing description is meant to be taken only by way of example and not to otherwise limit the scope of the present invention as defined in the appended claims.

I claim:

1. An apparatus comprising:
a first tube having a lumen;
a second tube having a lumen that is coupled to the first tube to extend alongside the first tube;
an inflatable balloon including a valve that is detachably coupled to a distal end of the second tube such that a portion of the distal end of the second tube extends through the valve;
wherein the balloon is adapted to be introduced into a space in a body and inflated in the space in the body by fluid introduced through the valve through the second tube; and
wherein the second tube is detachable from the inflated balloon to leave the inflated balloon positioned in the space in the body.

2. The apparatus according to claim 1, wherein the space in the body is a sub-pectoral space in a human patient.

3. The apparatus according to claim 1, wherein the space in the body is a sub-gluteal space in a human patient.

4. The apparatus according to claim 1, wherein the valve is self-sealing.

5. The apparatus according to claim 1, wherein the second tube is screwed into the balloon at a position of the valve to secure the distal end of the second tube extending through the valve.

6. A method comprising:
percutaneously inserting a needle inside of a cannula into a patient such that a distal end of the needle reaches a predetermined space in the body;
removing the needle and introducing a guide wire through the cannula;
removing the cannula;
inserting a catheter over the guide wire, the catheter having a balloon at a distal end thereof that is inflatable via an inflation catheter extending along side the catheter;
inflating the balloon, and detaching the balloon from the catheter;
extracting the catheter to leave the inflated balloon positioned in the space in the body.

7. The apparatus according to claim 6, wherein the space in the body is a sub-pectoral space in a human patient.

8. The apparatus according to claim 6, wherein the space in the body is a sub-gluteal space in a human patient.

9. An apparatus for lifting a breast, comprising:
a bone anchor adapted to be screwed into a rib;
a tissue anchor including a plurality of barbs to be lodged in breast tissue; and
a suture to connect the tissue anchor to the bone anchor;
wherein a length of the suture is set to a desired length to lift the breast by a desired amount.
